(19) United States
(12) Patent Application Publication
(10) Pub. No.: US 2006/0211664 A1
(43) Pub. Date: Sep. 21, 2006

(54) Method for treating erectile dysfunction and increasing libido in men

(76) Inventor: Robert E Dudley, Kenilworth, IL (US)

Correspondence Address: MAYER, BROWN, ROWE & MAW LLP, P.O. BOX 2828, CHICAGO, IL, 60690-2828, US

(21) Appl. No.: 10/531,526

(22) PCT Filed: Oct. 16, 2003
(86) PCT No.: PCT/US03/32597

§ 371 Date: Feb. 8, 2006

(30) Foreign Application Priority Data
Oct. 18, 2002 (US) 10273484

(51) Int. Cl. A61K 31/513; A61K 31/519

(52) U.S. Cl. 514/171; 514/252.16; 514/262.1

(57) ABSTRACT

The present invention relates to a transdermal hydroalcoholic testosterone gel formulation that overcomes the problems associated with other testosterone delivery mechanisms by providing, among other things, a desirable pharmacokinetic hormone profile with little or no skin irritation. In addition, the gel is used in conjunction with pharmaceuticals aimed at treating erectile dysfunction, such as VIAGRA©, to enhance their effectiveness.

### FIELD OF THE INVENTION

The present invention is directed to method of treating erectile dysfunction and increasing libido in men.

### BACKGROUND OF THE INVENTION

A. Sexual Performance, Erectile Dysfunction (“ED”), and Libido in Men

1. Sexual Performance & ED

“Sexual performance” as used herein generally refers to a man's ability to have an orgasm, obtain an erection, or engage in masturbation or intercourse. “Impotence” is a type of deficient sexual performance. Impotence or “erectile dysfunction” as used herein is generally refers to the inability of a man to attain an erection with sufficient rigidity for vaginal penetration 25% or more of the times attempted.

As many as 45 million men have some degree of erectile dysfunction. At least 10 million American men—about 9% of the adult population—are thought to have impotence. The rate increases with age. Thus, impotence affects about 10% of men in their sixties, 25% of men in their seventies, 40% of men in their eighties, and more than half of those in their nineties. In young couples, the incidence of impotence is about 7%. One-third of older men receiving medical treatment also have difficulty with erectile function.

Over the past decade, the medical perspective on the causes of impotence has shifted. Conventional wisdom used to attribute almost all cases of impotence to psychological factors. Investigators now estimate that between 70% and 80% of impotence cases are caused primarily by medical problems. Risk factors for impotence include hypogonadism, atherosclerosis, hypertension, diabetes mellitus, depression and other emotional or psychological illnesses, pelvic surgery, kidney failure, multiple sclerosis, stroke, some types of epilepsy, and alcoholism. Another risk factor is taking any of a variety of drugs, including cardiovascular medications, drugs that affect the central nervous system, certain hormonal preparations, heroin, and cocaine.

Today, 90% of all impotence cases are treated with VIAGRA® (sildenafil citrate USP). Other drugs useful in the treatment of impotence include, but are not limited to: pentoxifylline (TRENTAL®), yohimbine hydrochloride (ACTIBINE®, YOCON®, YOHIMEX®), apomorphine (UPRIMA®), alprostadil (the MUSE® system, TOPIGLAN®, CAVERJECT®), papavaerine (PAVABID®, CERESPAN®), and phentolamine (VASOMAX®, REGITINE®). In one embodiment, apomorphine is administered orally in a dose of about 2 mg to about 3 mg.

These pharmaceuticals act by a variety of physiological mechanisms. For example, the physiologic mechanism of erection of the penis involves release of nitric oxide (“NO”) in the corpus cavernosum during sexual stimulation. NO then activates the enzyme guanylate cyclase, which results in increased levels of cyclic guanosine monophosphate (“cGMP”), producing smooth muscle relaxation in the corpus cavemosum and allowing inflow of blood. VIAGRA® has no direct relaxant effect on isolated human corpus cavernosum, but enhances the effect of NO by inhibiting phosphodiesterase type 5 (“PDE5”), which is responsible for degradation of cGMP in the corpus cavernosum. When sexual stimulation causes local release of NO, inhibition of PDE5 by sildenafil causes increased levels of cGMP in the corpus cavernosum, resulting in smooth muscle relaxation and inflow of blood to the corpus cavernosum. In contrast, UPRIMA® is a dopamine receptor agonist that acts on the central nervous system. Once absorbed and transported into the brain, UPRIMA® initiates a chain of reactions that result in increased blood flow to the male genital organs and an erection. In accordance with the present invention, testosterone plays a beneficial role physiologically, and stimulates both sexual motivation (i.e., libido) and sexual performance.

2. Sexual Motivation and Libido

While the terms “sexual performance” and “impotence” describe physiological effects, the terms “sexual motivation” and “libido” describe psychological effects. “Libido” or “sexual motivation” as used herein is a parameter measured by the duration, frequency and extent of sexual daydreams, anticipation of sex, flirting, and sexual interaction.

As discussed above, while doctors now believe that erectile dysfunction is primarily caused by a physiological mechanism, some cases are still attributable to psychological causes. Moreover, decreased libido may also be a reaction to the experience of impotence. Unfortunately, pharmaceuticals such as VIAGRA® treat erectile dysfunction by the focusing on the physiological mechanics of attaining and maintaining an erection and do little or nothing to enhance the sexual motivation or libido of men suffering from erectile dysfunction. Thus, there remains a need to treat sexual performance disorders such as impotence in a manner that overcomes both the physiological and psychological problems associated with the disorder.

An number of clinical studies involving testosterone replacement in hypogonadal males have provided convincing evidence that testosterone plays a role in both sexual motivation libido and sexual performance. For example, researchers have reported that testosterone replacement results in increased sexual fantasies, sexual arousal and desire, spontaneous erections during sleep and in the morning, ejaculation, sexual activities with and without a partner, and orgasm through coitus or masturbation. See generally Christiansen, Behavioral Correlates of Testosterone, TESTOSTERONE: ACTION, DEFICIENCY, SUBSTITUTION 109-111 (1998).

B. Testosterone Synthesis, Metabolism, and Regulation

Testosterone, the major circulating androgen in men, is synthesized from cholesterol. The approximately 500 million Leydig cells in the testes secrete more than 95% of the 6-7 mg of testosterone produced per day. Two hormones produced by the pituitary gland, luteinizing hormone (“LH”) and follicle stimulating hormone (“FSH”), are required for the development and maintenance of testicular function and negatively regulate testosterone production. Circulating testosterone is metabolized to various 17-keto steroids through two different pathways. Testosterone can be metabolized to dihydrotestosterone (“DHT”) by the enzyme 5α-reductase or to estradiol (“E₂”) by an aromatase enzyme complex.

Testosterone circulates in the blood 98% bound to protein. In men, approximately 40% of the binding is to the high-affinity sex hormone binding globulin (“SHBG”). The remaining 60% is bound weakly to albumin. Thus, a number of measurements for testosterone are available from clinical laboratories. The term “free” testosterone as used herein refers to the fraction of testosterone in the blood that is not bound to protein. The term “total testosterone” or “testosterone” as used herein means the free testosterone plus protein-bound testosterone. The term “bioavailable testosterone” as used herein refers to the non-SHBG bound testosterone and includes testosterone weakly bound to albumin.

The following table from the UCLA-Harbor Medical Center summarizes the hormone concentrations in normal adult men range:

TABLE 1
Hormone Levels in Normal Men

| Hormone | Normal Range |
|---|---|
| Testosterone | 298 to 1043 ng/dL |
| Free testosterone | 3.5 to 17.9 ng/dL |
| DHT | 31 to 193 ng/dL |
| DHT/T Ratio | 0.052 to 0.33 |
| DHT + T | 372 to 1349 ng/dL |
| SHBG | 10.8 to 46.6 nmol/L |
| FSH | 1.0 to 6.9 mlU/mL |
| LH | 1.0 to 8.1 mlU/mL |
| E₂ | 17.1 to 46.1 pg/mL |

There is considerable variation in the half-life of testosterone reported in the literature, ranging from 10 to 100 minutes. Researchers do agree, however, that circulating testosterone has a diurnal variation in normal young men. Maximum levels occur at approximately 6:00 to 8:00 a.m. with levels declining throughout the day. Characteristic profiles have a maximum testosterone level of 720 ng/dL and a minimum level of 430 ng/dL. The physiological significance of this diurnal cycle, if any, however, is not clear.

C. Testosterone Levels and Sexual Behavior/Performance

Because increasing testosterone concentrations has been shown to alter sexual performance and libido, researchers have investigated methods of delivering testosterone to men. These methods include intramuscular injections (43%), oral replacement (24%), pellet implants (23%), and transdermal patches (10%). A summary of these methods is shown in Table 2.

TABLE 2
Mode of Application and Dosage of Various Testosterone Preparations

| Preparation | Route Of Application | Full Substitution Dose |
|---|---|---|
| In Clinical Use | | |
| Testosterone enanthate | Intramuscular injection | 200-25.0 g every 2-3 weeks |
| Testosterone cypionate | Intramuscular injection | 200 mg every 2 weeks |
| Testosterone undecanoate | Oral | 2-4 capsules at 40 mg per day |
| Transdermal testosterone patch | Scrotal skin | 1 membrane per day |
| Transdermal testosterone patch | Non-scrotal skin | 1 or 2 systems per day |
| Testosterone implants | Implantation under the | 3-6 implants of 200 mg every 6 |
| | abdominal skin | months |
| Under Development | | |
| Testosterone cyclodextrin | Sublingual | 2.5-5.0 mg twice daily |
| Testosterone undecanoate | Intramuscular injection | 1000 mg every 8-10 weeks |
| Testosterone buciclate | Intramuscular injection | 1000 mg every 12-16 weeks |
| Testosterone microspheres | Intramuscular injection | 315 mg for 11 weeks |
| Obsolete | | |
| 17α-Methyltestosterone | Oral | 25-5.0 g per day |
| Fluoxymesterone | Sublingual | 10-25 mg per day |
| | Oral | 10-20 mg per day |

All of the testosterone replacement methods currently employed, however, suffer from one or more drawbacks. For example, subdermal pellet implants and ester injections are painful and require doctor visits. Many of these methods, such as oral/sublingual/buccal preparations, suffer from undesirable pharmacokinetic profile—creating supra-physiologic testosterone concentrations followed a return to baseline. Transdermal patches provide less than optimal pharmacokinetic characteristics, are embarrassing for many patients, and are associated with significant skin irritation. Thus, although the need for an effective testosterone replacement methodology has existed for decades, an alternative replacement therapy that overcomes these problems has never been developed.

### SUMMARY OF THE INVENTION

The present invention relates to a transdermal hydroalcoholic testosterone gel formulation that overcomes the problems associated with other testosterone delivery mechanisms by providing, among other things, a desirable pharmacokinetic hormone profile with little or no skin irritation. The gel is used in conjunction with pharmaceuticals aimed at treating erectile dysfunction, such as VIAGRA®, to enhance their effectiveness.

### DETAILED DESCRIPTION OF THE INVENTION

While the present invention may be embodied in many different forms, several specific embodiments are discussed herein with the understanding that the present disclosure is to be considered only as an exemplification of the principles of the invention, and it is not intended to limit the invention to the embodiments illustrated. Where the invention is illustrated herein with particular reference to testosterone, it will be understood that any other steroid in the testosterone synthetic pathway can, if desired, be substituted in whole or in part for testosterone in the methods, kits, combinations, and compositions herein described. Where the invention is illustrated herein with particular reference to sildenafil, it will be understood that any other pharmaceutical agent for treating erectile dysfunction can, if desired, be substituted in whole or in part for sildenafil in the methods, kits, combinations, and compositions herein described.

The present invention is directed to methods, kits, combinations, and compositions for improving sexual performance in a subject, for example, a male subject, in need thereof. The method comprises delivering to the subject a pharmacologically effective amount of a steroid in the testosterone synthetic pathway in conjunction with a pharmaceutical agent for treating erectile dysfunction. In one embodiment, the present invention is directed to a method, kit, combination or pharmaceutical composition for percutaneous administration of a steroid in the testosterone synthetic pathway, for example, testosterone, in a hydroalcoholic gel useful for treating erectile dysfunction or libido deficiencies. The gel comprises one or more lower alcohols, such as ethanol or isopropanol; a penetration enhancing agent; a thickener; and water. Additionally, the present invention may optionally include salts, emollients, stabilizers, antimicrobials, fragrances, and propellants.

The present invention also includes kits, methods, combinations, and pharmaceutical compositions for reversing, halting or slowing the progression of sexual dysfunction in subject once it becomes clinically evident, or treating the symptoms associated with, or related to the sexual dysfunction. The subject may already have a sexual dysfunction at the time of administration, or be at risk of developing sexual dysfunction.

In one embodiment, the pharmaceutical composition of the present invention is administered once, twice, or three times a day, or as many times necessary to achieve the desired therapeutic effect. In another embodiment the composition of the present invention is administered once, twice, or three times a day on alternate days. In another embodiment the composition of the present invention is administered once, twice, or three times a day on a weekly, biweekly, or monthly basis.

A class of steroids in the testosterone synthetic pathway useful in the methods, kits, combinations, and compositions of the present invention include steroids in the testosterone anabolic or catabolic pathway. In a broad aspect of the invention, the active ingredients employed in the present invention may include anabolic steroids such as androisoxazole, androstenedione, bolasterone, clostebol, ethylestrenol, formyldienolone, 4-hydroxy-19-nortestosterone, methenolone, methyltrienolone, nandro lone, oxymesterone, quinbolone, stenbolone, trenbolone; androgenic steroids such as boldenone, dehydroepiandrosterone, fluoxymesterone, mestanolone, mesterolone, methandrostenolone, 17 alpha-methyltestosterone, 17 alpha-methyl-testosterone 3-cyclopentyl enol ether, norethandrolone, normethandrone, oxandrolone, oxymetholone, prasterone, stanlolone, stanozolol, dihydrotestosterone, testosterone; and progestogens such as anagestone, chlormadinone acetate, delmadinone acetate, demegestone, dimethisterone, dihydrogesterone, ethinylestrenol, ethisterone, ethynodiol, ethynodiol diacetate, flurogestone acetate, gestodene, gestonorone caproate, haloprogesterone, 17-hydroxy-16-methylene-progesterone, 17 alpha-hydroxyprogesterone, 17 alpha-hydroxyprogesterone caproate, medrogestone, medroxyprogesterone, megestrol acetate, melengestrol, norethindrone, norethindrone acetate, norethynodrel, norgesterone, norgestimate, norgesirel, norgestrienone, 19-norprogesterone, norvinisterone, pentagestrone, prenenolone, progesterone, promegestone, quingestrone, and trengestone; and all salts, esters, amides, enantiomers, isomers, tautomers, prodrugs and derivatives of these compounds. (Based in part upon the list provided in The Merck Index, Merck & Co. Rahway, N.J. (1998)). Combinations of the above mentioned steroids can be used in the methods, kits, combinations, and compositions herein described.

In one embodiment of the present invention, the steroid in the testosterone synthesis pathway is administered in conjunction with another pharmaceutical agent for treating erectile dysfunction, for example, an agent effective at inhibiting the activity of phosphodiesterase, as part of a specific treatment regimen intended to provide a beneficial effect from the co-action of these therapeutic agents for the treatment of a sexual disorder in a subject (“combination therapy”). The beneficial effect of the combination includes, but is not limited to, pharmacokinetic or pharmacodynamic co-action resulting from the combination of therapeutic agents, and also, for example, improving sexual performance such as treating erectile dysfunction and increasing libido in a subject. Administration of these therapeutic agents in combination typically is carried out over a defined time period (usually simultaneously, minutes, hours, days, weeks, months or years depending upon the combination selected). Combination therapy generally is not intended to encompass the administration of two or more of these therapeutic agents as part of separate monotherapy regimens that incidentally and arbitrarily result in the combinations of the present invention. Combination therapy is intended to embrace administration of these therapeutic agents in a sequential manner, that is, where each therapeutic agent is administered at a different time, as well as administration of these therapeutic agents, or at least two of the therapeutic agents, in a substantially simultaneous manner. Substantially simultaneous administration can be accomplished, for example, by administering to the subject a single gel having a fixed ratio of each therapeutic agent or in multiple, single capsules, tablets, or gels for each of the therapeutic agents. Sequential or substantially simultaneous administration of each therapeutic agent can be effected by any appropriate route including, but not limited to, an oral route, a percutaneous route, an intravenous route, an intramuscular route, or by direct absorption through mucous membrane tissues such as by an intranasal route.

The therapeutic agents can be administered by the same route or by different routes. For example, a first therapeutic agent of the combination selected may be administered orally, while the other therapeutic agents of the combination may be administered percutaneously. Alternatively, for example, all therapeutic agents may be administered percutaneously, or all therapeutic agents may be administered intravenously, or all therapeutic agents may be administered intramuscularly, or all therapeutic agents can be administered by direct absorption through mucous membrane tissues. The sequence in which the therapeutic agents are administered is not narrowly critical. Combination therapy also can embrace the administration of the therapeutic agents as described above in further combination with other biologically active ingredients, such as, but not limited to, another steroid or other pharmaceutical agents that increase testosterone levels in a subject, and non-drug therapies, such as, but not limited to, surgery.

A class of steroids or pharmaceutical agents that increases testosterone levels in a subject useful in the methods, kits, combinations, and compositions of the present invention include compounds that inhibit the synthesis of the sex hormone binding globulin. Sex hormone binding globulin is a serum protein, and is believed to bind to testosterone and estradiol, affecting the biological activity of these hormones. Specific compounds of interest that inhibit the synthesis the sex hormone binding globulin include but are not limited to methyltestosterone and fluoxymesterone, and all salts, esters, amides, enantiomers, isomers, tautomers, prodrugs and derivatives of these compounds. Combinations of the above these compounds can be used in the methods, kits, combinations, and compositions herein described. Methyltestosterone is currently available in various formulations including those available orally, for example, ANDROID® and TESTRED®. Fluoxymesterone is also currently available in various formulations including those available orally, for example, HALOSTESTIN®.

The therapeutic compounds which make up the combination therapy may be a combined dosage form or in separate dosage forms intended for substantially simultaneous administration. The therapeutic compounds that make up the combination therapy may also be administered sequentially, with either therapeutic compound being administered by a regimen calling for two step administration. Thus, a regimen may call for sequential administration of the therapeutic compounds with spaced-apart administration of the separate, active agents. The time period between the multiple administration steps may range from, for example, substantially simultaneous, or a few seconds or minutes to several hours to days, depending upon the properties of each therapeutic compound such as potency, solubility, bioavailability, plasma half-life and kinetic profile of the therapeutic compound, as well as depending upon the effect of food ingestion and the age and condition of the subject. Circadian variation of the target molecule concentration may also determine the optimal dose interval. In one embodiment, the steroid of the testosterone pathway is administered within about 24 or 48 hours before the pharmaceutical agent for treating erectile dysfunction. In another embodiment, the pharmaceutical agent for treating erectile dysfunction is administered within at least one day after the steroid of the testosterone pathway.

The therapeutic compounds of the combined therapy whether administered simultaneously, substantially simultaneously, or sequentially, may involve, for example, a regimen calling for administration of one therapeutic compound by oral route or intranasal route and another therapeutic compound by percutaneous route. Whether the therapeutic compounds of the combined therapy are administered orally, by inhalation spray, intranasal, rectally, topically, buccally (e.g., sublingual), or parenterally (e.g., subcutaneous, intramuscular, intravenous and intradermal injections, or infusion techniques), separately or together, each such therapeutic compound will be contained in a suitable pharmaceutical formulation of pharmaceutically-acceptable excipients, diluents or other formulations components. Examples of suitable pharmaceutically-acceptable formulations containing the therapeutic compounds are provided herein. Additionally, drug formulations are discussed in, for example, Hoover, John E., Remington's Pharmaceutical Sciences, Mack Publishing Co., Easton, Pa. 1975. Another discussion of drug formulations can be found in Liberman, H. A. and Lachman, L., Eds., Pharmaceutical Dosage Forms, Marcel Decker, New York, N.Y., 1980.

Besides being useful for human treatment, the present invention is also useful for veterinary treatment of mammals, reptiles, birds, exotic animals and farm animals, including mammals, rodents, and the like. In one embodiment, the mammal includes a primate, for example, a human, a monkey, or a lemur, a horse, a dog, a pig, or a cat. In another embodiment, the rodent includes a rat, a mouse, a squirrel or a guinea pig.

The methods, kits, combinations, and compositions of the present invention provide enhanced treatment options for treating sexual dysfunction in a subject, for example, a man, as compared to those currently available.

Included in the methods, kits, combinations and pharmaceutical compositions of the present invention are the isomeric forms and tautomers of the described compounds and the pharmaceutically-acceptable salts thereof. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, b-hydroxybutyric, galactaric and galacturonic acids.

In another embodiment of the present invention, the steroid of the testosterone pathway and the pharmaceutical agent for treating erectile dysfunction comes in the form of a kit or package containing one or more of the therapeutic compounds of the present invention. These therapeutic compounds of the present invention can be packaged in the form of a kit or package in which hourly, daily, weekly, or monthly (or other periodic) dosages are arranged for proper sequential or simultaneous administration. The present invention further provides a kit or package containing a plurality of dosage units, adapted for successive daily administration, each dosage unit comprising at least one of the therapeutic compounds of the present invention. This drug delivery system can be used to facilitate administering any of the various embodiments of the therapeutic compounds of the present invention. In one embodiment, the system contains a plurality of dosages to be to be administered daily or weekly. The kit or package can also contain the agents utilized in combination therapy to facilitate proper administration of the dosage forms. The kits or packages also contain a set of instructions for the subject.

In yet another embodiment, the present invention employs a packet having a polyethylene liner compatible with the components of a testosterone gel, as described below. The packet may hold a unit dose or multiple dose. In another embodiment, the methods, kits, combinations, and compositions employ a composition that is dispensed from a rigid multi-dose container (for example, with a hand pump) having a larger foil packet, for example, of the composition inside the container. Such larger packets can also comprise a polyethylene liner as above.

The term “prodrug” refers to a drug or compound in which the pharmacological action (active curative agent) results from conversion by metabolic processes within the body. Prodrugs are generally considered drug precursors that, following administration to a subject and subsequent absorption, are converted to an active or a more active species via some process, such as a metabolic process. Other products from the conversion process are easily disposed of by the body. Prodrugs generally have a chemical group present on the prodrug which renders it less active and/or confers solubility or some other property to the drug. Once the chemical group has been cleaved from the prodrug the more active drug is generated. Prodrugs may be designed as reversible drug derivatives and utilized as modifiers to enhance drug transport to site-specific tissues. The design of prodrugs to date has been to increase the effective water solubility of the therapeutic compound for targeting to regions where water is the principal solvent. For example, Fedorak, et al., Am. J. Physiol, 269:G210-218 (1995), describe dexamethasone-beta-D-glucuronide. McLoed, et al., Gastroenterol., 106:405-413 (1994), describe dexamethasone-succinate-dextrans. Hochhaus, et al., Biomed. Chrom., 6:283-286 (1992), describe dexamethasone-21-sulphobenzoate sodium and dexamethasone-21-isonicotinate. Additionally, J. Larsen and H. Bundgaard [Int. J. Pharmaceutics, 37, 87 (1987)] describe the evaluation of N-acylsulfonamides as potential prodrug derivatives. J. Larsen et al., [Int. J. Pharmaceutics, 47, 103 (1988)] describe the evaluation of N-methylsulfonamides as potential prodrug derivatives. Prodrugs are also described in, for example, Sinkula et al., J. Pharm. Sci., 64:181-210 (1975). Other nonlimiting examples of “prodrugs” that can be used in the combinations and methods of the present invention include parecoxib (propanamide, N-[[4-(5-methyl-3-phenyl-4-isoxazolyl)phenyl]sulfonyl]-), and MAG-camptothecin.

The term “derivative” refers to a compound that is produced from another compound of similar structure by the replacement of substitution of one atom, molecule or group by another. For example, a hydrogen atom of a compound may be substituted by alkyl, acyl, amino, etc., to produce a derivative of that compound.

The phrase “penetration enhancing agent” refers to an agent that accelerates the delivery of the drug through the skin. These agents also are referred to as accelerants, adjuvants, and absorption promoters, and are collectively referred to herein as “enhancers.” This class of agents includes those with diverse mechanisms of action including those which have the function of improving the solubility and diffusibility of the drug, and those which improve percutaneous absorption by changing the ability of the stratum corneum to retain moisture, softening the skin, improving the skin's permeability, acting as penetration assistants or hair-follicle openers or changing the state of the skin such as the boundary layer. The penetration enhancing agent of the present invention is a functional derivative of a fatty acid, which includes isosteric modifications of fatty acids or non-acidic derivatives of the carboxylic functional group of a fatty acid or isosteric modifications thereof. In one embodiment, the functional derivative of a fatty acid is an unsaturated alkanoic acid in which the —COOH group is substituted with a functional derivative thereof, such as alcohols, polyols, amides and substituted derivatives thereof. The term “fatty acid” means a fatty acid that has four (4) to twenty-four (24) carbon atoms.

Non-limiting examples of penetration enhancing agents include C8-C22 fatty acids such as isostearic acid, octanoic acid, and oleic acid; C8-C22 fatty alcohols such as oleyl alcohol and lauryl alcohol; lower alkyl esters of C8-C22 fatty acids such as ethyl oleate, isopropyl myristate, butyl stearate, and methyl laurate; di(lower)alkyl esters of C6-C22 diacids such as diisopropyl adipate; monoglycerides of C8-C22 fatty acids such as glyceryl monolaurate; tetrahydrofurfuryl alcohol polyethylene glycol ether; polyethylene glycol, propylene glycol; 2-(2-ethoxyethoxy)ethanol; diethylene glycol monomethyl ether; alkylaryl ethers of polyethylene oxide; polyethylene oxide monomethyl ethers; polyethylene oxide dimethyl ethers; dimethyl sulfoxide; glycerol; ethyl acetate; acetoacetic ester; N-alkylpyrrolidone; and terpenes.

The thickening agents, or gelling agents, used herein may include anionic polymers such as polyacrylic acid (CARBOPOL® by B. F. Goodrich Specialty Polymers and Chemicals Division of Cleveland, Ohio), carboxypolymethylene, carboxymethylcellulose and the like, including derivatives of Carbopol® polymers, such as Carbopol® Ultrez 10, Carbopol® 940, Carbopol® 941, Carbopol® 954, Carbopol® 980, Carbopol® 981, Carbopol® ETD 2001, Carbopol® EZ-2 and Carbopol® EZ-3, and other polymers such as Pemulen® polymeric emulsifiers, and Noveon® polycarbophils. Additional thickening agents, enhancers and adjuvants may generally be found in Remington's The Science and Practice of Pharmacy, Meade Publishing Co., United States Pharmacopeia/National Formulary.

As used herein, the term “lower alcohol,” alone or in combination, means a straight-chain or branched-chain alcohol moiety containing one to about six carbon atoms. In one embodiment, the lower alcohol contains one to about 4 carbon atoms, and in another embodiment the lower alcohol contains two to about 3 carbon atoms. Examples of such alcohol moieties include methanol, ethanol, n-propanol, isopropanol, n-butanol, isobutanol, sec-butanol, and tert-butanol.

As used herein, the term “lower alkyl”, alone or in combination, means a straight-chain or branched-chain alkyl radical containing one to about six carbon atoms. In one embodiment, the lower alkyl contains one to about four carbon atoms. Examples of such radicals include methyl, ethyl, n-propyl, isopropyl, n-butyl, isobutyl, sec-butyl, and tert-butyl.

The composition is used in a “pharmacologically effective amount.” This means that the concentration of the drug administered is such that in the composition it results in a therapeutic level of drug delivered over the term that the drug is to be used. Such delivery is dependent on a number of variables including the time period for which the individual dosage unit is to be used, the flux rate of the drug from the composition, for example, testosterone, from the gel, surface area of application site, etc. For testosterone, for example, the amount of testosterone necessary can be experimentally determined based on the flux rate of testosterone through the gel, and through the skin when used with and without enhancers.

Illustratively, certain formulations of the present invention deliver about 0.01 g to about 100 g testosterone, or the equivalent thereof, to a subject per dosage unit. In another embodiment of the present invention, the formulations deliver from about 0.1 g to about 10 g testosterone, or the equivalent thereof, to a subject per dosage unit. In yet another embodiment of the present invention, the formulations of the present invention deliver from about 0.17 g to about 5 g testosterone, or the equivalent thereof, to a subject per dosage unit. In another embodiment of the present invention, the formulations of the present invention deliver about 1 g testosterone, or the equivalent thereof, to a subject per dosage unit. In still another embodiment of the present invention, the formulations of the present invention deliver about 0.25 g testosterone, or the equivalent thereof, to a subject per dosage unit. Thus, for example, a testosterone gel, ointment, cream or patch is formulated as a single dosage unit for once a day administration contains about 0.17 g, or about 0.25 g, or about 0.5 g testosterone, or about 1.0 g testosterone, while a gel, ointment, cream or patch formulated as a single dosage unit for once a week administration contains about 1.19 g, or about 1.75 g, or about 3.50 g, or about 7.0 g testosterone, respectfully.

In one embodiment, the formulation is a gel, an ointment, a cream or a patch and is comprised of testosterone; a penetration enhancing agent, such as isopropyl myristate; a thickening agent, such as Carbopol; a lower alcohol, such as ethanol or isopropanol; and water. In another embodiment the formulation is a gel, an ointment, a cream or a patch and is comprised of the following substances in approximate percentages:

TABLE 3
Composition of Testosterone Formulation

| SUBSTANCE | AMOUNT (w/w) |
|---|---|
| Testosterone | 0.01-70% |
| Penetration enhancing agent | 0.01-50% |
| Thickening agent | 0.01-50% |
| Lower alcohol | 30-98% |
| Purified water (qsf) | 100% |

Illustratively, in a 100 g composition, the gel, ointment, cream, or patch may contain about 0.01 g to about 70 g of testosterone, about 0.01 g to about 50 g penetration enhancing agent, about 0.1 g to about 50 g thickening agent, and about 30 g to about 98 g lower alcohol. In another embodiment, in a 100 g composition, the gel, ointment, cream, or patch may contain about 0.1 g to 10 g of testosterone, about 0.1 g to about 5 g of penetration enhancing agent, about 0.1 g to about 5 g of thickening agent, an about 45 g to about 90 g lower alcohol.

In one embodiment, the composition is a gel, ointment, cream, or patch that further comprises a hydroxide releasing agent, such as sodium hydroxide (for example, 0.1 N NaOH), in an amount of about 0.1% to about 10% w/w of the composition.

In another embodiment, the pharmaceutical composition includes about 0.5% to about 10% testosterone; about 30% to about 98% alcohol, for example, ethanol or isopropanol; about 0.1% to about 5% isopropyl myristate; about 1% to about 5% sodium hydroxide; and about 0.1% to about 5% of a gelling agent. The percentages of components are weight to weight of the composition.

In yet another embodiment, the pharmaceutical composition includes testosterone in a hydroalcoholic gel. The testosterone may be present in a concentration of about 0.5%, 1%, 2%, 3%, 4%, 5%, 6%, 7%, 8%, 9%, or 10% weight to weight of the composition. The enhancer in this embodiment includes isopropyl myristate, which may be present in a concentration of about 0.5%, 1%, 2%, 3%, 4%, or 5% weight to weight of the composition. The pharmaceutical composition also includes a C1-C4 alcohol present in a concentration of about 72.5% weight to weight of the composition. Further, the pharmaceutical composition includes polyacrylic acid and/or carboxymethylcellulose as the gelling agent. In one embodiment, the gelling agent is polyacrylic acid present in a concentration of about 1% weight to weight of the composition.

One such testosterone gel has only recently been made available in the United States under the trademark AndroGel® by Unimed Pharmaceuticals, Inc., Marietta, Ga., the assignee of this application. In one embodiment, the gel is comprised of the following substances in approximate amounts:

TABLE 4
Composition of AndroGel ®

| | AMOUNT (w/w) | |
|---|---|---|
| SUBSTANCE | PER 100 g OF GEL | |
| Testosterone | 1.0 | g |
| Carbopol 980 | 0.90 | g |
| Isopropyl myristate | 0.50 | g |
| 0.1 N NaOH | 4.72 | g |
| Ethanol (95% w/w) | 72.5 | g* |
| Purified water (qsf) | 100 | g |

*Corresponding to 67 g of ethanol.

One skilled in the art will appreciate that the constituents of this formulation may be varied in amounts yet continue to be within the spirit and scope of the present invention. For example, the composition may contain about 0.1 to about 10.0 g of testosterone, about 0.1 to about 5.0 g CARBOPOL, about 0.1 to about 5.0 g isopropyl myristate, and about 30.0 to about 98.0 g ethanol.

In still another embodiment, the composition comprises testosterone in an amount greater than 0.01%, a penetration enhancing agent in an amount greater than about 0.1%, a thickening agent in an amount greater than about 0.1%, and a lower alcohol in an amount greater than about 30% w/w of the composition.

The gel, ointment, cream, or patch is rubbed or placed onto an area of skin of the subject and allowed to dry. Illustratively, the gel, ointment, or cream is rubbed onto an area of skin, for example, on the upper outer thigh and/or hip once daily. Following application the subject washes his or her hands. Application of the gel results in an increased testosterone level having a desirable pharmacokinetic profile effective to treat or prevent sexual dysfunction, or the symptoms associated with, or related to sexual dysfunction in the subject. The composition is thus useful for treating a number of sexual dysfunctions, disorders, conditions or diseases in both men and women.

In one embodiment of the present invention a method is provided for treating, preventing sexual dysfunction in a subject in need thereof, that is, a subject indicated for having, or at risk of developing sexual dysfunction. The method comprises administering a pharmacologically effective amount of a composition to an area of skin of the subject for delivery of a steroid in the testosterone synthetic pathway to blood serum of the subject. The composition comprises:

(a) about 0.01% to about 70% (w/w) steroid in the testosterone synthetic pathway;

(b) about 0.01% to about 50% (w/w) penetration enhancing agent;

(c) about 0.01% to about 50% (w/w) gelling agent; and

(d) about 30% to about 98% (w/w) lower alcohol.

The composition is capable of releasing the steroid after applying the composition to the skin at a rate and duration that delivers in one embodiment of the present invention at least about 10 μg per day of the steroid to the blood serum of the subject.

In one embodiment of the present invention the steroid in the testosterone synthetic pathway is testosterone.

In another embodiment of the methods, kits, combinations, and compositions of the present invention, the composition is capable of releasing the testosterone after applying the composition to the skin of a subject at a rate and duration that achieves a circulating serum concentration of testosterone greater than about 400 ng per dl serum during a time period beginning about 2 hours after administration and ending about 24 hours after administration.

In another embodiment of the methods, kits, combinations, and compositions of the present invention, the composition is capable of releasing the testosterone after applying the composition to the skin of a subject at a rate and duration that achieves a circulating serum concentration of the testosterone between about 400 ng testosterone per dl serum to about 1050 ng testosterone per dl serum.

In another embodiment of the methods, kits, combinations, and compositions of the present invention, for each about 0.1 gram per day application of the composition of the present invention to the skin of a subject, an increase of at least about 5 ng/dl in serum testosterone concentration results in the subject.

In another embodiment of the methods, kits, combinations, and compositions of the present invention, the composition of the present invention is provided to a subject for daily administration in about a 0.1 g to about a 10 g dose.

In yet another embodiment of the methods, kits, combinations, and compositions of the present invention, the subject in need of treatment has a serum testosterone level before the first application (pretreatment) of the composition of the present invention of less than about 300 ng/dl.

In another embodiment of the methods, kits, combinations, and compositions of the present invention, where after at least about 30 days of daily administration of the composition of the present invention the serum testosterone concentration in a subject is at least about 490 ng/dl to about 860 ng/dl.

In still another embodiment of the methods, kits, combinations, and compositions of the present invention, where after at least about 30 days of daily administration of the composition of the present invention the total serum androgen concentration in a subject is greater than about 372 ng/dl.

In another embodiment of the methods, kits, combinations, and compositions of the present invention, the composition of the present invention is administered once, twice, or three times daily to a subject for at least about 7 days.

The present invention also provides a method of treating, preventing or reducing the risk of developing sexual dysfunction in a subject in need thereof, that is, a subject indicated for having, or at risk of developing sexual dysfunction, by administering to the subject:

(a) an amount of a composition comprising:
(i) about 0.01% to about 70% (w/w) steroid in the testosterone synthetic pathway;
(ii) about 0.01% to about 50% (w/w) penetration enhancing agent;
(iii) about 0.01% to about 50% (w/w) thickening agent; and
(iv) about 30% to about 98% (w/w) lower alcohol; and

(b) an amount of a therapeutic agent for treating erectile dysfunction. The composition is administered to an area of skin of the subject for delivery of the steroid in the testosterone synthetic pathway to the blood serum of the subject, and is capable of releasing the steroid after applying the composition to the skin at a rate and duration that delivers at least about 10 μg per day of the steroid to the blood serum of the subject. The amount of the composition and the amount of the therapeutic agent together make a pharmacologically effective amount.

In one embodiment of the methods, kits, combinations, and compositions of the present invention, the composition and the therapeutic agent are provided as separate components to a kit.

In another embodiment of the methods, kits, combinations, and compositions of the present invention, the composition and the therapeutic agent are administered substantially simultaneously, or sequentially.

In still another embodiment of the methods, kits, combinations, and compositions of the present invention, the therapeutic agent is administered orally, percutaneously, intravenously, intramuscularly, or by direct absorption through mucous membrane tissue.

The present invention also provides a pharmaceutical composition, comprising:

(i) about 0.01% to about 70% (w/w) steroid in the testosterone synthetic pathway;

(ii) about 0.01 % to about 50% (w/w) penetration enhancing agent;

(iii) about 0.01% to about 50% (w/w) thickening agent;

(iv) about 30% to about 98% (w/w) lower alcohol; and

(v) a therapeutic agent for treating erectile dysfunction.

The composition is administered to an area of skin of the subject for delivery of the testosterone and the therapeutic agent to the blood serum of the subject, and is capable of releasing the steroid after applying the composition to the skin at a rate and duration that delivers at least about 10 μg per day of the steroid to the blood serum of the subject. The amount of the testosterone and the amount of the therapeutic agent together make an amount sufficient to treat erectile dysfunction in a subject.

Achieving target delivery rates demonstrated by testosterone gel can be estimated from the pharmacokinetics in testosterone gel in men. The mean serum concentration (Cavg) values in men after applying of varying amounts of gel to the upper body is given in the Table below.

TABLE 5
Mean Average Serum Testosterone Concentrations and Daily Delivery
Rate after Administration of Testosterone Gel 1% in Men

| Dose (μL) | Mean Cavg | Daily Delivery Rate |
|---|---|---|
| (gram) | (ng/dL) | (μg/day)^{a} |
| 5.0 | 555 (±225) | 3330 |
| 7.5 | 601 (±309) | 3606 |
| 10 | 713 (±209) | 4278 |

^{a}Metabolic Clearance Rate of Daily Testosterone = 600 L/day

Based on the results obtained in men, a testosterone gel dose of 0.5 grams delivers approximately 300 μg of testosterone per day.

Toxicity and therapeutic efficacy of the active ingredients can be determined by standard pharmaceutical procedures, e.g., for determining LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects

It has been shown, and is discussed in co-pending U.S. application Ser. No. 09/703,753, that transdermal application of testosterone using AndroGel® to hypogonadal men results in improved libido and sexual performance. AndroGel® may also be used in combination with pharmaceuticals useful for treating erectile dysfunction. Such pharmaceuticals include any agent that is effective to inhibit the activity of a phosphodiesterase. Suitable phosphodiesterase inhibitors include, but are not limited to, inhibitors of the type III phosphodiesterase (cAMP-specific-cGMP inhibitable form), the type IV phosphodiesterase (high affinity-high specificity cAMP form) and the type V phosphodiesterase (the cGMP specific form). Additional inhibitors that may be used in conjunction with the present invention are cGMP-specific phosphodiesterase inhibitors other than type V inhibitors.

Examples of type III phospodiesterase inhibitors that may be administered include, but are not limited to, bypyridines such as milrinone and amirinone, imidazolones such as piroximone and enoximone, dihydropyridazinones such as imazodan, 5-methyl-imazodan, indolidan and ICI1118233, quinolinone compounds such as cilostamide, cilostazol and vesnarinone, and other molecules such as bemoradan, anergrelide, siguazodan, trequinsin, pimobendan, SKF-94120, SKF-95654, lixazinone and isomazole.

Examples of type IV phosphodiesterase inhibitors suitable herein include, but are not limited to, rolipram and rolipram derivatives such as RO20-1724, nitraquazone and nitraquazone derivatives such as CP-77059 and RS-25344-00, xanthine derivatives such as denbufylline and ICI63197, and other compounds such as EMD54622, LAS-31025 and etazolate.

Examples of type V phosphodiesterase inhibitors include, but are not limited to, zaprinast, MY5445, dipyridamole, vardenafil and sildenafil. Other type V phosphodiesterase inhibitors are disclosed in PCT Publication Nos. WO 94/28902 and WO 96/16644. In the preferred embodiment, an inhibitor of phosphodiesterase type 5 (“PDE5”), such as VIAGRA® (sildenafil citrate USP) is administered in an amount of about 25 mg to 200 mg. In one embodiment, sildenafil citrate is administered orally in a dose of about 25 mg, 50 mg, or 100 mg. In another embodiment, sildenafil citrate is administered intranasally in an amount of about 10 mg, 20 mg, or 40 mg. By example, U.S. Pat. No. 6,200,591 discloses the intranasal administration of sildenafil.

The compounds described in PCT Publication No. WO 94/28902 are pyrazolopyrimidinones. Examples of the inhibitor compounds include 5-(2-ethoxy-5-morpholinoacetylphenyl)-1-methyl-3-n-propyl-1,6-dihydro-7H-pyrazolo[4,3-d]pyrimidin-7-one, 5-(5-morpholinoacetyl-2-n-propoxyphenyl)-1-methyl-3-n-propyl-1,6-dihydro-7-H-pyrazolo[4,3-d]pyrimidin-7-one, 5-[2-ethoxy-5-(4-methyl-1-piperazinylsulfonyl)-phenyl]1-methyl-3-n-propyl-1,6-dihydro-7H-pyrazolo[4,3-d]pyrimidin-7-one, 5-[2-allyloxy-5-(4-methyl-1-piperazinylsulfonyl)-phenyl]-1-methyl-3-n-propyl-1,6-dihydro-7H-pyrazolo[4,3-d]pyrimidin-7-one, 5-[2-ethoxy-5-[4-(2-propyl)-1-piperazinylsulfonyl)-phenyl]-1-methyl-3-n-propyl-1,6-dihydro-7H-pyrazolo[4,3-d]pyrimidin-7-one, 5-[2-ethoxy-5-[4-(2-hydroxyethyl)-1-piperazinylsulfonyl)phenyl]-1-methyl-3-n-propyl-1,6-dihydro-7H-pyrazolo[4,3-d]pyrimidin-7-one, 5[5-[4-(2-hydroxyethyl)-1-piperazinylsulfonyl]-2-n-propoxyphenyl]-1-methy 1-3-n-propyl-1,6-dihydro-7H-pyrazolo[4,3-d]pyrimidin-7-one, 5-[2-ethoxy-5-(4-methyl-1-piperazinylcarbonyl)phenyl]-1-methyl-3-n-propyl-1,6-dihydro-7H-pyrazolo[4,3-d]pyrimidin-7-one, and 5-[2-ethoxy-5-(1-methyl-2-imidazolyl)phenyl]-1-methyl-3-n-propyl-1,6-dihydro-7H-pyrazolo[4,3-d]pyrimidin-7-one.

The phosphodiesterase inhibitors described in PCT Publication No. WO 96/16644 include griseolic acid derivatives, 2-phenylpurinone derivatives, phenylpyridone derivatives, fused and condensed pyrimidines, pyrimidopyrimidine derivatives, purine compounds, quinazoline compounds, phenylpyrimidinone derivative, imidazoquinoxalinone derivatives or aza analogues thereof, phenylpyridone derivatives, and others. Specific examples of the phosphodiesterase inhibitors disclosed in WO 96/16644 include 1,3-dimethyl-5-benzylpyrazoio[4,3-d]pyrimidine-7-one, 2-2-propoxyphenyl)-6-purinone, 6-(2-propoxyphenyl)-1,2-dihydro-2-oxypyridine-3-carboxamide, 2-(2-propoxyphenyl)-pyrido[2,3-d]pyrimid-4(3H)-one, 7-methylthio-4-oxo-2-(2-propoxyphenyl)-3,4-dihydro-pyrimido[4,5-d]pyrimidine, 6-hydroxy-2-(2-propoxyphenyl)pyrimidine4-carboxamide, 1-ethyl-3-methylirnidazo[1,5a]quinoxalin-4(5H)-one, 4-phenylmethylamino-6-chloro-2-(1-imidazoloyl)quinazoline, 5-ethyl-8-[3-(N-cyclohexyl-N-methylcarbamoyl)-propyloxy]-4,5-dihydro-4-oxo-pyrido[3,2-e]-pyrrolo[1,2-a]pyrazine, 5′-methyl-3′-(phenylmethyl)-spiro[cyclopentane-1,7′(8′H)-(3′H)-imidazo[2,1b]purin]4′(5′H)-one, 1-[6-chloro-4-(3,4-methylenedioxybenzyl)-aminoquinazolin-2-yl)piperidine-4-carboxylic acid, (6R, 9S)-2-(4-trifluoromethyl-phenyl)methyl-5-methyl-3,4,5,6a,7,8,9,9a-octahydrocyclopent[4,5]-midazo[2,1-b]-purin-4-one, 1t-butyl-3-phenylmethyl-6-(4-pyridyl)pyrazolo[3,4-d]-pyrimid-4-one, 1-cyclopentyl-3-methyl-6-(4-pyridyl)-4,5-dihydro-1H-pyrazolo[3,4-d]pyrimid-4-one, 2-butyl-1-(2-chlorobenzyl)6-ethoxy-carbonylbenzimidaole, and 2-(4-carboxypiperidino)-4-(3,4-methylenedioxy-benzyl)amino-6-nitroquinazoline, and 2-phenyl-8-ethoxycycloheptimidazole.

Still other type V phosphodiesterase inhibitors useful in conjunction with the present invention include: IC-351 (ICOS); 4-bromo-5-(pyridylmethylamino)-6-[3-(4-chlorophenyl)propoxy]-3(2H)pyridazinone; 1-[4-[(1,3-benzodioxol-5-ylmethyl)amiono]-6-chloro-2-quinazolinyl]-4-piperidine-carboxylic acid, monosodium salt; (+)-cis-5,6a,7,9,9,9a-hexahydro-2-[4-(trifluoromethyl)-phenymmethyl-5-methyl-cyclopent-4,5]imidazo[2,1-b]purin-4(3H)one; furazlocillin; cis-2-hexyl-5-methyl-3,4,5,6a,7,8,9,9a-octahydrocyclopent[4,5]imidazo[2,1-b]purin-4-one; 3-acetyl-1-(2-chlorobenzyl)-2-propylindole-6-carboxylate; 4-bromo-5-(3-pyridylmethylamino)-6-(3-(4-chlorophenyl)propoxy)-3-(2H)pyridazinone; 1-methyl-5-(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)amino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No.5064 (Kyowa Hakko; see WO 96/26940); Pharmaprojects No. 5069 (Schering Plough); GF-196960 (Glaxo Wellcome); Sch-59496; Sch-51866; KF-31327 (Kyowa Hakko); N2-isonicotinylpyrroloquinolone PDE V inhibitors (Johnson and Johnson); B carboline derivatives (Johnson and Johnson); UK-369003 (Pfizer); NCX-911 (NicOx); DA-8159 (Dong-A); FR-229934 (Fujisawa); TA-1790 (Tanabe Seiyaku); NMI-870 (NitroMed); PT-141 (Palatin Technologies); AWD-12171 (Viatris); BMS-223131 (Bristol-Myers Squibb); E-8010 (Eisai); LAS-34179 (Almirall-Prodesfarma); PNU-83757 (Pharmacia); ABT-598 (Abbott); PG-005 (F-Gene); EMR-6203 (Merck); and moxisylyte hydrochloride (Viatris);.

Other phosphodiesterase inhibitors that may be used in the method of this invention include nonspecific phosphodiesterase inhibitors such as theophylline, IBMX, pentoxifylline and papaverine, and direct vasodilators such as hydralazine.

The pharmaceutical or therapeutic agents for treating erectile dysfunction may be administered, if desired, in the form of salts, esters, amides, prodrugs, derivatives, and the like, provided the salt, ester, amide, prodrug or derivative is suitable pharmacologically, i.e., effective in the present method. Salts, esters, amides, prodrugs and other derivatives of the active agents may be prepared using standard procedures known to those skilled in the art of synthetic organic chemistry and described, for example, by J. March, Advanced Organic Chemistry; Reactions, Mechanisms and Structure, 4th Ed. (New York: Wiley-Interscience, 1992). For example, acid addition salts are prepared from the free base using conventional methodology, and involves reaction with a suitable acid. Generally, the base form of the drug is dissolved in a polar organic solvent such as methanol or ethanol and the acid is added thereto. The resulting salt either precipitates or may be brought out of solution by addition of a less polar solvent. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Particularly preferred acid addition salts of the active agents herein are halide salts, such as may be prepared using hydrochloric or hydrobromic acids. Conversely, preparation of basic salts of acid moieties which may be present on a phosphodiesterase inhibitor molecule are prepared in a similar manner using a pharmaceutically acceptable base such as sodium hydroxide, potassium hydroxide, ammonium hydroxide, calcium hydroxide, trimethylamine, or the like. Particularly preferred basic salts herem are alkali metal salts, e.g., the sodium salt, and copper salts. Preparation of esters involves functionalization of hydroxyl and/or carboxyl groups which may be present within the molecular structure of the drug. The esters are typically acyl-substituted derivatives of free alcohol groups, i.e., moieties which are derived from carboxylic acids of the formula RCOOH where R is alkyl, and preferably is lower alkyl. Esters can be reconverted to the free acids, if desired, by using conventional hydrogenolysis or hydrolysis procedures. Amides and prodrugs may also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine. Prodrugs are typically prepared by covalent attachment of a moiety, which results in a compound that is therapeutically inactive until modified by an individual's metabolic system.

Other compounds useful for treating erectile dysfunciton may also be used. These include: (a) pentoxifylline (TRENTAL®); (b) yohimbine hydrocholoride (ACTIBINE®, YOCON®, YOHIMEX®; (c) apomorphine (UPRIMA®); (d) alprostadil (the MUSE® system, TOPIGLAN®, CAVERJECT®; (e) papavaerine (PAVABID®, CERESPAN®); (f) phentolamine (VASOMAX®, REGITINE®), and combinations, salts, derivatives and enantiomers of all of the above.

A testosterone containing gel, such as AndroGel® is administered to increase and enhance the therapeutic effectiveness of such drugs, in either hypogonadal or eugonadal men having erectile dysfunction. While pharmaceuticals such as VIAGRA® work principally by various physiological mechanisms of erection initiation and maintenance, the testosterone gel used in accordance with the present invention plays a beneficial role physiologically, and stimulates both sexual motivation (i.e., libido) and sexual performance. Testosterone controls the expression of the nitric oxide synthase gene. See Reilly et al., Androgenic Regulation of NO Availability in Rat Penile Erection, 18 J. ANDROLOGY 110 (1997); Park et al., Effects of Androgens on the Expression of Nitric Oxide Synthase mRRNAs in Rat Corpous Cavernosum, 83 BJU INT'L. 327 (1999). Thus, testosterone and other androgens clearly play a role in erectile dysfunction. See Lugg et al., The Role of Nitric Oxide in Erectile Function, 16 J. ANDROLOGY 2 (1995); Penson et al., Androgen and Pituitary Control of Penile Nitric Oxide Synthase and Erectile Function In the Rat, 55 BIOLOGY OF REPRODUCTION 576 (1996); Traish et al., Effects of Castration and Androgen Replacement on Erectile Function in a Rabbit Model, 140 ENDOCRINOLOGY 1861 (1999). Moreover, testosterone replacement restores nitric oxide activity. See Baba et al. Delayed Testosterone Replacement Restores Nitric Oxide Synthase Containing Nerve Fibres and the Erectile Response in Rat Penis, BJU INT'L 953 (2000); Garban et al., Restoration of Normal Adult Penile Erectile Response in Aged Rats by Long-Term Treatment with Androgens, 53 BIOLOGY OF REPRODUCTION 1365 (1995); Marin et al., Androgen-dependent Nitric Oxide Release in Rat Penis Correlates with Levels of Constitutive Nitric Oxide Synthase Isoenzymes, 61 BIOLOGY OF REPRODUCTION 1012 (1999).

As disclosed herein, adequate blood levels of testosterone are important to erection. In one embodiment, AndroGel® is applied to the body in accordance with the protocol summarized in Example 1. The pharmaceutical(s) for erectile dysfunction is taken in accordance with the prescription requirements. For example, VIAGRA® is generally taken 20-40 minutes before sexual intercourse in 50 mg doses. This combination of therapy is particularly useful in hypogonadal men who need increased testosterone levels in order to optimize the effects of VIAGRA® and the sexual experience as a whole. In essence, a therapeutic effect is obtained. AndroGel® is preferably applied to the body for a sufficient number of days so that the steady-state levels of testosterone are achieved.

The present invention is further illustrated by the following examples, which should not be construed as limiting in any way. The contents of all cited references throughout this application are hereby expressly incorporated by reference. The practice of the present invention will employ, unless otherwise indicated, conventional techniques of pharmacology and pharmaceutics, which are within the skill of the art.

### EXAMPLES

### Example 1

### Testosterone Gel Plus Sildenafil Improves Sexual Performance in Sildenafil Non-Responders

One embodiment of the present invention involves the transdermal application of a testosterone gel co-administered with an oral dose of sildenafil as a method of producing an erectile response in hypogonadal men who do not respond to treatment with sildenafil alone for erectile dysfunction.

In this example, hypogonadal men who did not respond to sildenafil alone in the treatment of erectile dysfunction were recruited and studied in several centers across the United States. The study was double-blind for a testosterone gel 1% (Androgel®) and a placebo gel. The mean age of the patients was 58.5 years. Selection criteria for patients included: erectile dysfunction for at least the past 3 months, involvement in a stable heterosexual relationship, nonresponsive to 100 mg of sildenafil (a score of 2 or 3 on each of Questions 3 and 4 of the International Index of Erectile Function (IIEF), see below), and low to low normal testosterone serum levels (<400 ng/dL collected before 10:00 am). The IIEF is a brief, reliable, self-administered questionnaire of erectile function utilized in cross cultural settings for detecting treatment-related changes in patients. The IIEF consists of 15 questions directed to individual sexual performance. Each question includes 6 possible responses (0-5, 0 representing non-performance, generally, and 5 representing no indication, generally). Based on a principal components analysis of the score, five factors or response domains are identified: (1) Erectile Function (EF); (2) Orgasmic Function (OF); (3) Sexual Desire (SD); (4) Intercourse Satisfaction (IS); and (5) OveralI Satisfaction (OS).

A total of 75 patients were enrolled and randomized to receive 5.0 g/day of Androgel® (delivering 50 mg/day of testosterone to the skin of which about 10% or 5 mg is absorbed) plus 100 mg of sildenafil (1 hour before intercourse) or 5.0 g/day placebo gel plus 100 mg of sildenafil (1 hour before intercourse). The subjects applied 5.0 g/day of the Androgel® or placebo gel to clean dry skin of the shoulders, upper arms, and/or abdomen and orally ingested 100 mg/day of sildenafil. The patients were treated for 12 weeks. An interim analysis on 67 subjects at Week 4 showed that Androgel® significantly improved response to sildenafil on EF, OF, and OS domains, and IIEF Total Score over the placebo gel. The primary outcome measures included the mean change from baseline (BL) in the Erectile Function domain of the IIEF. Secondary outcome measures included the mean change from baseline in each of the remaining four domains and total score of the IIEF. Safety assessments included a physical exam, urologic exam, PSA, vital signs, laboratory tests and adverse events.

The following table summarizes the IIEF outcome measures:

TABLE 6
IIEF Outcome Measures

| | Change from BL (Mean ± SD) | | |
|---|---|---|---|
| IIEF Domain | Androgel ® + Sildenafil | Placebo + Sildenafil | P* |
| Erectile Func. | 5.65 ± 6.66 | 2.97 ± 5.13 | 0.037 |
| Orgasmic Func. | 1.53 ± 2.38 | 0.36 ± 2.03 | 0.019 |
| Sexual Desire | 0.44 ± 2.02 | 0.00 ± 1.68 | 0.211 |
| Intercourse Satis. | 1.21 ± 2.33 | 0.70 ± 1.94 | 0.250 |
| Overall Satis. | 1.62 ± 2.26 | 0.61 ± 1.98 | 0.046 |
| Total Score | 10.44 ± 13.21 | 4.64 ± 9.88 | 0.022 |

*Based on ANOVA with treatment and pooled center as fixed effects.

As shown in Table 6, testosterone replacement therapy with testosterone-gel improves erectile response to sildenafil and may be utilized in the treatment of erectile dysfunction in men with low to low-normal testosterone who failed prior treatment with sildenafil alone. Although the AndroGel® 1% testosterone gel formulation was employed in this study, the present invention is not limited to only this one embodiment. Other embodiments may use higher or lower amounts of androgen, penetration enhancer(s) and excipients to achieve the present invention.

All cited literature and patent references are hereby incorporated herein by reference. Although the invention has been described with respect to specific embodiments and examples, it should be appreciated that other embodiments utilizing the concept of the present invention are possible without departing from the scope of the invention. The present invention is defined by the claimed elements, and any and all modifications, variations, or equivalents that fall within the true spirit and scope of the underlying principles.

I claim:

1. A method of improving sexual performance in a male subject, comprising:
(a) administering a pharmaceutical composition to skin of the subject, the composition comprising a pharmacologically effective amount of testosterone, a penetration enhancer, a C1-C4 alcohol, and a gelling agent forming a hydroalcoholic gel formulation; and
(b) administering a pharmacologically effective amount of a phosphodiesterase inhibitor to the subject after the administration of the gel formulation.

2. The method of claim 1, wherein the penetration enhancer comprises at least one of a C8-C22 fatty acid.

3. The method of claim 2, wherein the fatty acid comprises an alkyl chain length of at least 12 carbon atoms.

4. The method of claim 1, wherein the alcohol comprises at least one of ethanol, 2-propanol, n-propanol, or mixtures thereof.

5. The method of claim 1, wherein the inhibitor is administered in a single dose.

6. The method of claim 1, wherein the hydroalcoholic gel formulation is administered in a single dose or divided dose.

7. The method of claim 1, wherein the inhibitor is administered within about 24 hours after the administration of the hydroalcoholic gel formulation.

8. The method of claim 1, wherein the inhibitor is selected from the group consisting of a type III phosphodiesterase inhibitor, a type IV phosphodiesterase inhibitor, and a type V phosphodiesterase inhibitor.

9. The method of claim 8, wherein the inhibitor is a type V phosphodiesterase inhibitor selected from the group consisting of sildenafil, sildenafil citrate, zaprinast, MY5445, dipyridamole, and vardenafil, or an enantiomer, isomer, or salt thereof.

10. The method of claim 1, wherein the inhibitor is sildenafil citrate administered in an amount of about 25 mg to about 200 mg.

11. The method of claim 10, wherein the sildenafil citrate is administered in an amount of about 25 mg, 50 mg, or 100 mg.

12. The method of claim 1, wherein the inhibitor is administered via a route selected from the group consisting of oral, intranasal, inhalation, parenteral and percutaneous.

13. The method of claim 10, wherein the sildenafil citrate is administered orally in an amount of about 25 mg, 50 mg, or 100 mg.

14. The method of claim 12, wherein the sildenafil citrate is administered intranasally in an amount of about 10 mg, 20 mg, or 40 mg.

15. The method of claim 1, wherein the subject achieves hormonal steady state levels of testosterone.

16. The method of claim 1, wherein the subject is hypogonadal.

17. The method of claim 1, wherein the enhancer is isopropyl myristate.

18. The method of claim 17, wherein the isopropyl myristate is present in a concentration selected from the group consisting of about 0.5%, 1%, 2%, 3%, 4%, and 5% weight to weight of the composition.

19. The method of claim 18, wherein the isopropyl myristate is present in a concentration of about 0.5% weight to weight of the composition.

20. The method of claim 1, wherein the gelling agent is selected from the group consisting of polyacrylic acid, and carboxymethylcellulose.

21. The method of claim 1, wherein the gelling agent is polyacrylic acid present in a concentration of about 1% weight to weight of the composition.

22. The method of claim 1, wherein the alcohol is present in a concentration of about 72.5% weight to weight of the composition.

23. The method of claim 1, wherein the testosterone is present in a concentration selected from the group consisting of about 0.5%, 1%, 2%, 3%, 4%, 5%, 6%, 7%, 8%, 9%, and 10% weight to weight of the composition.

24. The method of claim 1, wherein the pharmaceutical composition further comprises sodium hydroxide.

25. The method of claim 1, wherein the pharmaceutical composition comprises:
(a) about 0.5% to about 10% testosterone;
(b) about 30% to about 98% alcohol selected from the group consisting of ethanol and isopropanol;
(c) about 0.1% to about 5% isopropyl myristate;
(d) about 1% to about 5% sodium hydroxide; and
(e) about 0.1% to about 5% of a gelling agent;
wherein the percentages of components are weight to weight of the composition.

26. The method of claim 1, wherein the composition is contained in a packet selected from the group consisting of a unit dose packet and a multiple dose packet.

27. A method of improving sexual performance in a male subject, comprising:
(a) administering a pharmaceutical composition to skin of the subject, the composition comprising a pharmacologically effective amount of testosterone, a penetration enhancer, a C1-C4 alcohol, and a gelling agent forming a hydroalcoholic gel formulation; and
(b) administering a pharmaceutical agent for treating erectile dysfunction to the subject after the administration of the gel formulation.

28. The method of claim 27, wherein the penetration enhancer comprises at least one of a C8-C22 fatty acid.

29. The method of claim 28, wherein the fatty acid comprises an alkyl chain length of at least 12 carbon atoms.

30. The method of claim 27, wherein the alcohol comprises at least one of ethanol, 2-propanol, or n-propanol, and mixtures thereof.

31. The method of claim 27, wherein the pharmaceutical agent for treating erectile dysfunction is administered in a single dose.

32. The method of claim 27, wherein the hydroalcoholic gel formulation is administered in a single dose or divided dose.

33. The method of claim 27, wherein the pharmaceutical agent for treating erectile dysfunction is administered within about 24 hours after the administration of the hydroalcoholic gel formulation.

34. The method of claim 27, wherein the pharmaceutical agent for treating erectile dysfunction is selected from the group consisting of pentoxifylline, yohimbine, apomorphine, alprostadil, papavaerine, and phentolamine, or a combination, salt, derivative or enantiomer thereof.

35. The method of claim 34, wherein the pharmaceutical agent for treating erectile dysfunction is apomorphine administered orally in an amount of about 2 mg to about 3 mg.

36. The method of claim 27, wherein the pharmaceutical agent for treating erectile dysfunction is administered via a route selected from the group consisting of oral, intranasal, inhalation, parenteral, and percutaneous.

37. The method of claim 27, wherein the subject achieves hormonal steady state levels of testosterone.

38. The method of claim 27, wherein the subject is hypogonadal.

39. The method of claim 27, wherein the enhancer is isopropyl myristate.

40. The method of claim 39, wherein the isopropyl myristate is present in a concentration selected from the group consisting of about 0.5%, 1%, 2%, 3%, 4%, and 5% weight to weight of the composition.

41. The method of claim 40, wherein the isopropyl myristate is present in a concentration of about 0.5% weight to weight of the composition.

42. The method of claim 27, wherein the gelling agent is selected from the group consisting of polyacrylic acid, and carboxymethylcellulose.

43. The method of claim 27, wherein the gelling agent is polyacrylic acid present in a concentration of about 1% weight to weight of the composition.

44. The method of claim 27, wherein the alcohol is present in a concentration of about 72.5% weight to weight of the composition.

45. The method of claim 27, wherein the testosterone is present in a concentration selected from the group consisting of about 0.5%, 1%, 2%, 3%, 4%, 5%, 6%, 7%, 8%, 9%, and 10% weight to weight of the composition.

46. The method of claim 27, wherein the pharmaceutical composition further comprises sodium hydroxide.

47. The method of claim 27, wherein the pharmaceutical composition comprises:
(a) about 0.5% to about 10% testosterone;
(b) about 30% to about 98% alcohol selected from the group consisting of ethanol and isopropanol;
(c) about 0.1% to about 5% isopropyl myristate;
(d) about 1% to about 5% sodium hydroxide; and
(e) about 0.1% to about 5% of a gelling agent;
wherein the percentages of components are weight to weight of the composition.

48. The method of claim 27, wherein the composition is contained in a packet selected from the group consisting of a unit dose packet, and a multiple dose packet.
